(19) United States
(12) Patent Application Publication
(10) Pub. No.: US 2011/0287549 A1
(43) Pub. Date: Nov. 24, 2011

(54) TIME TEMPERATURE INDICATOR BASED ON THIOALKYL AND THIOARYL SUBSTITUTED SPIROAROMATICS

(75) Inventors: Husein Salman, Golan Heights (IL); Elena Tenetov, Nesher (IL)

(73) Assignee: FRESHPOINT HOLDINGS SA, La Chaux-de-Fonds (CH)

(21) Appl. No.: 13/146,230

(22) PCT Filed: Feb. 9, 2010
(86) PCT No.: PCT/EP10/51528

§ 371 Date: Aug. 16, 2011

(30) Foreign Application Priority Data
Feb. 12, 2009 (EP) 09152675.6

(51) Int. Cl. G01N 21/78; B05D 3/02; G01D 21/00; C07D 491/107

(52) U.S. Cl. 436/92; 548/409; 427/591; 427/384; 116/201

(57) ABSTRACT

The present invention relates to photochromic spiropyrans as active ingredients of Time-Temperature Indicators (TTIs), and to new spiropyrans per se. More particularly, the invention provides TTIs on the base of photochromic spiropyrans comprising alkylsulfanyl/arylsulfanyl substituents in the phenyl ring of the benzopyrane moiety.

The present invention relates to photochromic spiropyrans as active ingredients of Time-Temperature Indicators (TTIs), and to new spiropyrans per se. More particularly, the invention provides TTIs on the base of photochromic spiropyrans comprising alkylsulfanyl/arylsulfanyl substituents in the phenyl ring of the benzopyrane moiety.

TTIs are substrates for packaging of or attachment to perishable goods that respond to cumulative exposure to time and temperature. TTIs are capable of reporting the partial or full time temperature history of any good to which it is thermally coupled. TTI relies on a spiroaromatic compounds which are reversibly photochromic. The compounds can undergo photo-induced coloration by irradiation with photons of a specific energy range (preferably UV light of near UV light), the coloration being followed by a time- and temperature-dependent decoloration. TTIs based on a photochromic compound should, ideally, not be affected by surrounding light. Unfortunately some undesirable simultaneous processes such as re-charging and photobleaching/photodegradation of the indicator compound can occur.

Therefore prevention of these undesirable processes is very important. In order to prevent the first process there are different filter systems, for example those disclosed in the international publication WO 2008/083895. However existing filters cannot ensure complete protection against photobleaching and/or photodegradation of the indicator compound. Stability of the indicator to the photobleaching/photodegradation processes is determined mostly by its inherent properties. Therefore search for new compounds with improved photostability is very important.

U.S. Pat. No. 4,286,957 discloses in column 7 in Example 7 “[dimethyl 3,3 isopropyl 1 indolino]2 spiro 2′ nitro 6 methyl thioisopropyl 8′ benzopyran” as photochromic ingredient of an optical lens. The nomenclature of this substance is rather strange (cf. inter alia the numbering of the atoms) and it is unclear which structure is meant by it. Possibly, the following structure is meant which is not encompassed within the definition of the compounds of the formula I of the present invention due to the isopropylthiomethyl group.

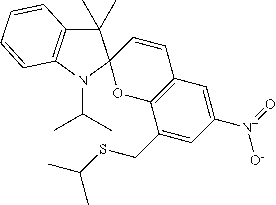

TTIs based on spiroaromatic photochromic compounds were described earlier.

WO 99/39197 describes the use of photochromic dyes, based on a transfer reaction as active materials for TTIs. TTIs based on these materials are highly accurate and reproducible and can be charged using stimulating light. It further teaches that by placing a special filter atop the active substance most of the UV and visible spectrum of light can be filtered which prevents undesired re-charging and photobleaching of the TTI.

The International Publications WO05/075978A2 (Freshpoint) and WO2008/083925A1 (Freshpoint) teach TTIs based on monomeric spiropyrans. However, most of the known monomeric spiropyrans are disadvantageous in that they are characterized by a relatively high bleaching. The compounds disclosed in WO05/075978A2 differ from the presently disclosed monomeric spiropyrans inter alia in that the definition of the substituent Y on page 15, lines 4-6 of WO05/075978A2 allows only for substitution of the aralkyl moiety by halogen and not by the substituent meanings listed herein below for the substituent R₇ of the present invention.

TTIs based on oligomeric spiropyrans with increased photostability and improved lifetimes are disclosed in the International Publication WO2008/090045A1 (Freshpoint). There is no disclosure of oligomeric thioalkyl- or thioaryl substituted spiroaromatics.

The problem underlying the present invention is therefore to provide a time-temperature indicator system having a good photostability allowing an effective and precise monitoring of the temperature of perishable products.

We have now found a series of photochromic compounds, namely, thioalkyl- and thioaryl-derivatives of spiropyrans possessing desirable properties for TTI applications. The derivatives develop a strong color in the solid state upon UV irradiation, some demonstrate extremely low photobleaching, high stability under storage at room temperature before activation and printability on commercially used substrates. The information drawn from the TTI based on the compounds of the series is highly accurate, reproducible and proportional to the time-temperature history of the perishable goods to which they are attached.

### SHORT DESCRIPTION OF THE FIGURES

FIGS. 1 to 4 compare the photobleaching of the thio-spiropyrans of the present invention with a sample kept in the dark and with the corresponding behaviour of structurally mots closely related oxy-spiropyrans belonging to the prior art.

FIGS. 1 to 4 show along the horizontal axis the time [hours] and along the vertical axis the square root of the sum of the square delta Lab values, which is calculated from the CIE L, a, and b values according to the formula (ΔL²+Δa²+Δb²)^{0.5}, wherein ΔL is L₀−L, Δa is a₀−a and Δb is b₀−b and L₀, a₀ and b₀ are the values at time 0, i.e. the time just before charging. A detailed description of the Figures and the conclusions drawn therefrom are provided in Example 5 of the present text.

The invention relates to new thioalkyl- and thioaryl-derivatives of spiropyrans of general formulae I, Ia, II, III and IV and to TTIs comprising these derivatives.

In one embodiment the invention relates to new thioalkyl- and thioaryl-derivatives of spiropyrans of the general formula I

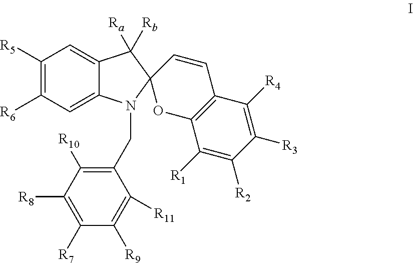

wherein
R₁-R₄ independently of one another is hydrogen, C₁-C₆ alkylsulfanyl, arylsulfanyl, halogen, CF₃, —C₁-C₆ alkyl or —NO₂ with the proviso that at least one of R₁-R₄ is C₁-C₆ alkylsulfanyl or arylsulfanyl, preferably C₁-C₆ alkylsulfanyl, especially C₁-C₂ alkylsulfanyl, e.g. C₁-alkylsulfanyl;
R₅ is hydrogen, halogen, —C₁-C₆ alkoxy, —COOH, —COO—C₁-C₆alkyl, —CF₃ or phenyl;
R₆ is hydrogen or R₆ and R₅ form together a phenyl ring;
Rₐ is hydrogen or, —C₁-C₆ alkyl;
R_{b} is hydrogen or, —C₁-C₆ alkyl, or together with Rₐ form a 5-6 membered cycle;
R₇ is —C₁-C₆ alkoxy, —NO₂, —CF₃, ₋O—CF₃, —CN, —COO—C₁-C₆alkyl, phenyl or biphenyl;
R₈ is hydrogen, halogen, —CN, —C₁-C₆ alkoxy or R₇ and R₈ form together a phenyl ring;
R₉ is hydrogen, halogen, —CN, or —C₁-C₆ alkoxy;
R₁₀ is hydrogen or halogen or CN;
R₁₁ is hydrogen or halogen or CN,
   and to TTIs comprising these derivatives.

In a preferred embodiment the present invention provides a compound of formula I wherein:
R₁ is C₁-C₆ alkylsulfanyl, arylsulfanyl, preferably C₁-C₆ alkylsulfanyl, especially C₁-C₂ alkylsulfanyl, e.g. C₁alkylsulfanyl;
R₂ and R₄ are hydrogen;
R₃ is NO₂,
R₅ and R₆ are hydrogen;
Rₐ and R_{b} are methyl;
R₇ is —C₁-C₆ alkoxy, —NO₂, —CF₃, ₋O—CF₃, —CN, —COO—C₁-C₆alkyl, phenyl or biphenyl, preferably, phenyl, biphenyl, more preferably biphenyl;
R₈ and R₉ and R₁₀ and R₁₁ are hydrogen.

Most preferred R₁ is C₁-C₆ alkylsulfanyl, especially MeS and R₇ is —NO₂, phenyl or biphenyl.

Preferably, C₁-C₆ alkylsulfanyl, especially C₁-C₆ alkylsulfanyl R₁, is different from isopropylsulfanyl, i.e. 2-propylsulfanyl, and is e.g. C₁-C₂ alkylsulfanyl or C₄-C₆ alkylsulfanyl, or 1-propylsulfanyl.

Examples are:

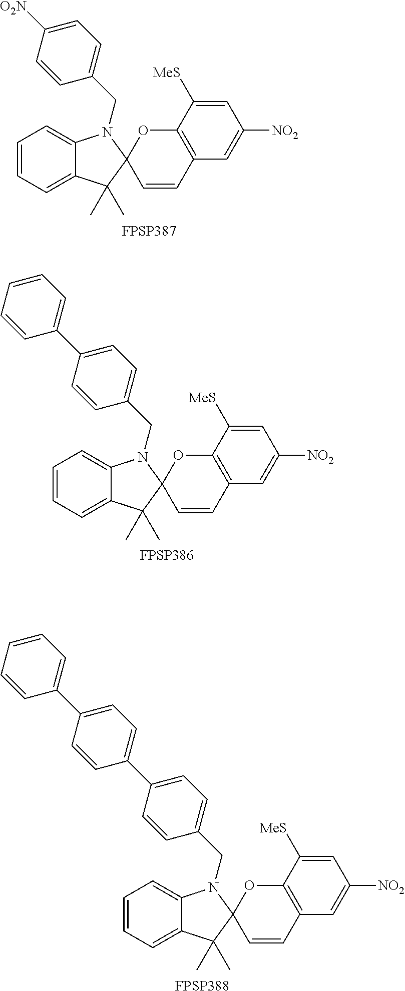

The International Publication WO05/075978 (Freshpoint) generically discloses spiroaromatic compounds

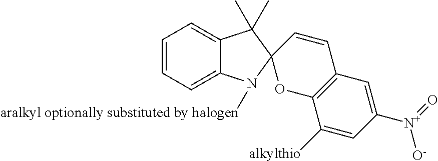

There are no specific examples of thioalkyl compounds disclosed in WO05/075978.

In one embodiment the invention relates to new thiomethyl- and thioaryl-derivatives of spiropyrans as a selection of the general disclosure of WO05/075978 of the general formula Ia

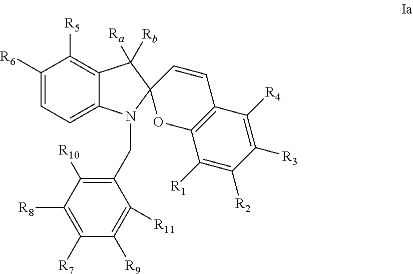

wherein
R₁ is MeS (=methylsulfanyl=methylthio) or arylsulfanyl, preferably MeS;
R₂ and R₄ are hydrogen;
R₃ is NO₂,
R₅ and R₆ are hydrogen;
Rₐ and R_{b} are methyl;
R₇ is hydrogen or halogen;
R₈ and R₉ and R₁₀ and R₁₁ are hydrogen, and to TTIs comprising these compounds.

Thus, resulting in compounds

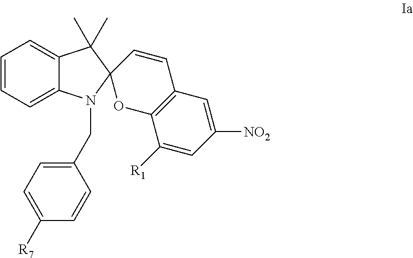

wherein R₁ is MeS or arylsulfanyl and R₇ is hydrogen or halogen.

Example is:

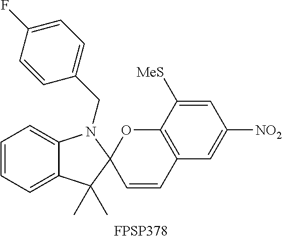

In one embodiment the invention relates to new oligomeric thioalkyl- and thioaryl-derivatives of spiropyrans of the general formula II or III

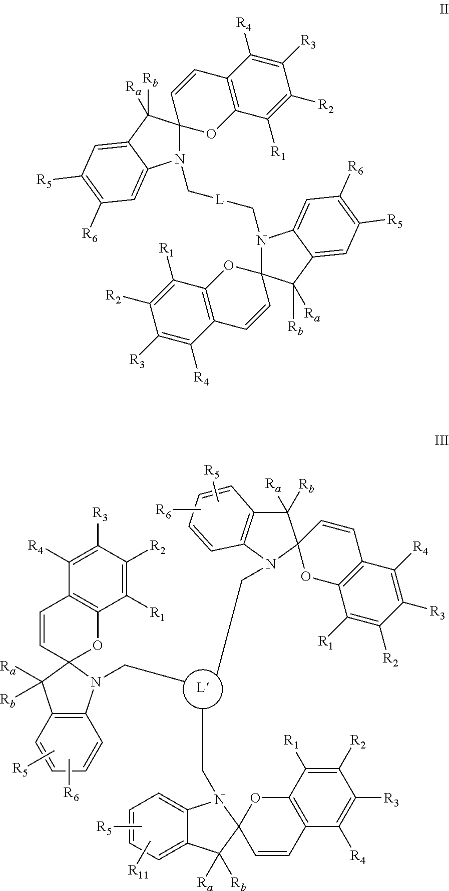

wherein
R₁-R₄ independently of one another is hydrogen, C₁-C₆ alkylsulfanyl, arylsulfanyl, halogen, CF₃, —C₁-C₆ alkyl or —NO₂ with the proviso that at least one of R₁-R₄ is C₁-C₆ alkylsulfanyl or arylsulfanyl, preferably C₁-C₆ alkylsulfanyl;
R₅ is hydrogen, halogen, —C₁-C₆ alkoxy, —COOH, —COO—C₁-C₆ alkyl, —CF₃ or phenyl;
R₆ is hydrogen or R₆ and R₅ form together a phenyl ring;
Rₐ is hydrogen or, —C₁-C₆ alkyl;
R_{b} is hydrogen or, —C₁-C₆ alkyl, or together with Rₐ forms a 5-6 membered ring
L is a divalent linker;
L′ is a trivalent linker, and to TTIs comprising these compounds.

In another preferred embodiment the invention provides a compound of formula II wherein
R₁ is —C₁-C₆ alkylsulfanyl or arylsulfanyl, preferably C₁-C₆ alkylsulfanyl;
R₂ and R₄ are hydrogen;
R₃ is NO₂
R₅ and R₆ are hydrogen;
Rₐ and R_{b} are methyl;
L is a divalent linker.

Examples are:

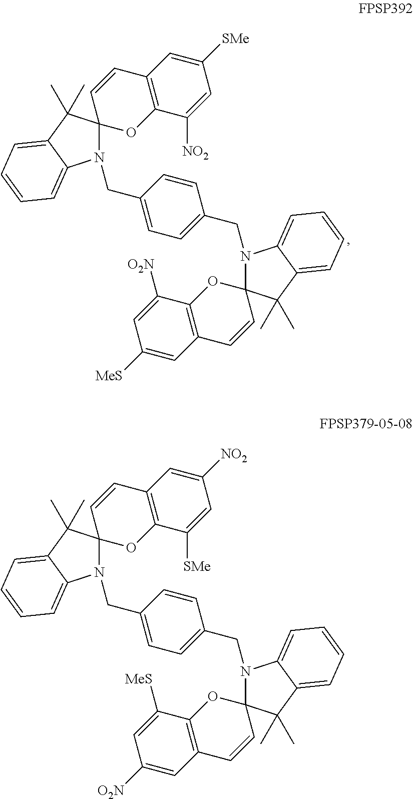

The invention relates especially to a time-temperature indicator, wherein the at least one thioalkyl derivative of the spiropyran indicator compound is selected from the group consisting of the following structural formulae

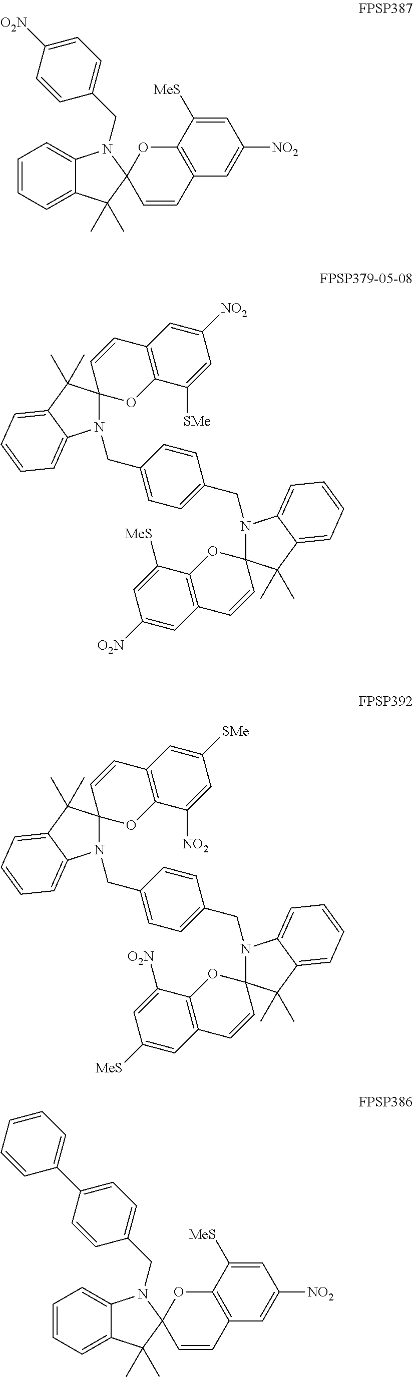

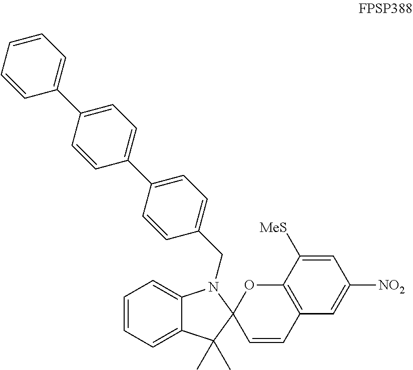

especially of FPSP387, FPSP379-05-08, FPSP386 and FPSP388, and to any one of these compounds per se.

In one embodiment the spiroaromatic compound is trimeric.

A spiropyran trimer of the formula III is for example

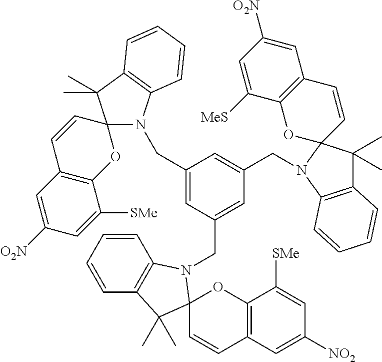

Concerning the linked spiroaromatic compounds of the formula II or III, those compounds of the formula II are preferred.

In one embodiment the invention relates to new thioalkyl- and thioaryl-derivatives of spiropyrans of the general formula IV.

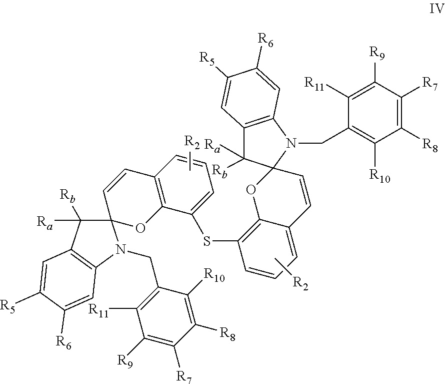

wherein
R₂ is hydrogen, halogen, CF₃, —C₁-C₆ alkyl or —NO₂,
R₅ is hydrogen, halogen, —C₁-C₆ alkoxy, —COOH, —COO—C₁-C₆ alkyl, —CF₃ or phenyl;
R₆ is hydrogen or R₆ and R₅ form together a phenyl ring;
Rₐ is hydrogen or, —C₁-C₆ alkyl;
R_{b} is hydrogen or —C₁-C₆ alkyl, or together with Rₐ forms a 5-6 membered ring.

The term “alkylsulfanyl,” as used herein, represents an alkyl group attached to the parent molecular moiety through a sulfur atom.

The term “arylsulfanyl,” as used herein, represents an aryl group attached to the parent molecular moiety through a sulfur atom. In this specification the generic term —C₁-C₆ alkyl includes both straight chain and branched chain alkyl groups such as propyl, iso-propyl and tert-butyl groups and also cycloalkyl(C₅-C₆) groups such as cyclopentyl, and cyclohexyl.

The term “divalent linker” or “trivalent linker” as used herein refers to any divalent or trivalent group capable of linking two or three spiropyran moieties together, e.g. a divalent or trivalent aliphatic or, preferably, aromatic group.

Examples of aliphatic divalent linker groups are selected from C₁-C₁₂ alkylene, C₁-C₁₂ alkenylene, and C₁-C₁₂ alkynylene. Examples of aromatic divalent linker groups are selected from

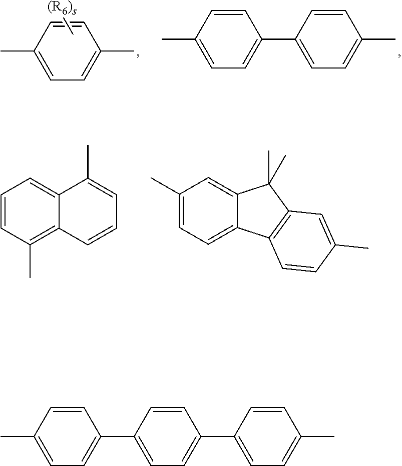

wherein R₆ is hydrogen, halogen, —C₁-C₆ alkoxy, CF₃, NO₂, preferably methoxy or hydrogen; and s is 1-4, preferably 1 or 2.

In one embodiment the linker is -phenyl-, like e.g. 1,4-phenylene.

Examples of trivalent linker groups are

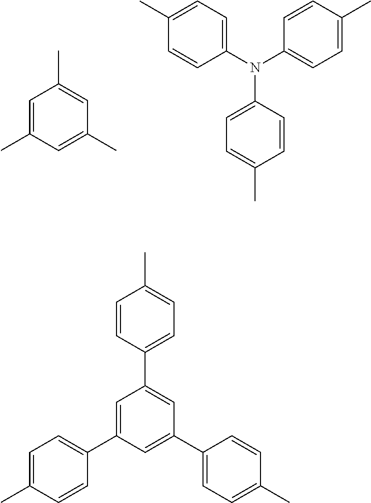

C₁-C₆ alkoxy is preferably methoxy. “Halogen” refers to fluoro, chloro, bromo or iodo.

In the above thioalkyl- or thioaryl spiropyrans there are at least two different metastable isomers. At least two distinct valence isomeric forms exist in each spiroaromatic unit of the oligomeric indicator. These isomeric forms are at least one colored open form, first isomeric form, and at least one colorless cyclic form (closed form or second isomeric form).

Suitable active materials exhibit the following characteristics:
(1) the system has at least one thermal process leading from one metastable state to one stable state, where the two states of the spiroaromatic compounds are characterized by a distinctly different color and/or any other measurable physical parameter such as luminescence, refraction index, conductivity and the like.
(2) the stable state may be converted into the metastable state using one or any combination of stimuli, among others the following processes:
   a) photonic induction,
   b) thermal induction,
   c) pressure induction,
   d) electrical induction, or
   e) chemical induction; and
(3) other than temperature and photoinduction (in the visible light range), the metastable state is substantially not affected by anyone or any combination of stimuli such as a) photo induction, b) piezo induction, c) electro induction, d) chemo induction.

Photoinduction means that the initially colourless indicator is irradiated with light, preferably in the UV or near-UV range, as a result a reversible internal valence isomerisation from a colourless inactivated state to a coloured activated one is induced. A reverse discolouration process then proceeds at a rate that is time and temperature dependent.

The metastable state may further be achieved by pressure induction. In this procedure, the matrix embedded with and/or atop the substance is passed between two bodies, such as metal rolls, which apply pressure onto the surface of the matrix thereby inducing the formation of the metastable state. By adjusting the time and pressure imparted by the bodies to the active material, it is possible to control the degree of conversion from a stable state to a metastable state in the TTI active matrix.

The metastable state may be achieved by thermal induction. In this particular induction process, the matrix embedded with the substance to be induced is heated to temperatures normally below the melting point of said substance. The heat may be applied by any method known such as, but not limited to, a thermal transfer printing head. In one specific case, the heat is applied to the matrix while being passed through two heated metal rolls. In this case, the pressure applied to the surface is not capable itself of inducing the formation of the meta-stable state, but serves merely to ensure controlled thermal contact between the heaters and the sample. The metastable state is achieved as a result of the heat transfer from the heaters, i.e., the metal rolls, which are in contact with the matrix and the matrix itself.

However, there may be instances where the use of any combination of pressure, light and thermal inductions may be desired or necessary. It is therefore, a further embodiment of the present invention, to achieve the metastable state of the substances to be used with the TTIs of the present invention, by a combination of stimuli.

The active material of the present invention may be in the form of a crystal or a poly-crystalline powder, in which the forward and reverse reactions take place or alternatively may be in a form of any other condensed phase such as a glass, a polymer solution or attached to a polymer, or in the form of a liquid or a solution.

In yet another aspect of the present invention, there is provided a method for the manufacture of a TTI comprising at least one of the thioalkyl- or thioaryl-spiroaromatic indicator compounds of the formula I, Ia, II, III or IV in form of a pigment or a dye; said method comprising the steps of
(a) introducing into a matrix or atop a matrix a thioalkyl- or thioaryl-spiroaromatic indicator compound of the formula I, Ia, II, III or IV as defined here in and
(b) converting the spiropyran indicator from an original stable state into a metastable state by a process selected from photonic induction, thermal induction, pressure induction, electrical induction, or chemical induction,
(c) optionally applying a protector film.

The converting step b may be effected immediately after step a) or later at any time.

The original stable state and the metastable state is defined above.

The term “introducing into a matrix” means any form of admixing the TTI indicator into a matrix, for example, indicator-doping of the matrix, sol-gel embedment of the indicator in the matrix, embedment of the indicator as small crystallites, solid solution and the like.

The matrix used in the present invention may be a polymer, an adhesive, all kinds of paper or cardboard, all kinds of printing media, metal, or any glass-like film.

The matrix is also called substrate.

Examples of printing media may be self-adhesive PP, cold lamination films, PVC films, PP paper, glossy photo paper, vinyl sheets and the like; inkjet media.

The matrix polymer is a high molecular weight organic material may be of natural or synthetic origin and generally has a molecular weight in the range of from 10³ to 10⁸ g/mol. It may be, for example, a natural resin or a drying oil, rubber or casein, or a modified natural material, such as chlorinated rubber, an oil-modified alkyd resin, viscose, a cellulose ether or ester, such as cellulose acetate, cellulose propionate, cellulose acetobutyrate or nitrocellulose, but especially a totally synthetic organic polymer (thermosetting plastics and thermoplastics), as are obtained by polymerisation, polycondensation or polyaddition, for example polyolefins, such as polyethylene, polypropylene or polyisobutylene, substituted polyolefins, such as polymerisation products of vinyl chloride, vinyl acetate, styrene, acrylonitrile, acrylic acid esters and/or methacrylic acid esters or butadiene, and copolymerisation products of the mentioned monomers, especially ABS or EVA. From the group of the polyaddition resins and polycondensation resins there may be mentioned the condensation products of form-aldehyde with phenols, so-called phenoplasts, and the condensation products of form-aldehyde with urea, thiourea and melamine, so-called aminoplasts, the polyesters used as surface-coating resins, either saturated, such as alkyd resins, or unsaturated, such as maleic resins, also linear polyesters and polyamides or silicones. The mentioned high molecular weight compounds may be present individually or in mixtures, in the form of plastic compositions or melts. They may also be present in the form of their monomers or in the polymerised state in dissolved form as film-forming agents or binders for surface-coatings or printing inks, such as boiled linseed oil, nitrocellulose, alkyd resins, melamine resins, urea-formaldehyde resins or acrylic resins.

The term “introducing” means also printing. In this case, the TTI is transformed into a printable ink.

The ink may directly be printed onto the packaging material or label.

Thus, the present invention further concerns a printing ink or printing ink concentrate, comprising at least one thioalkyl- or thioaryl-spiroaromatic indicator compounds of the formula I, Ia, II, III or IV as defined here in; for manufacturing a time temperature indicator. Any of the printing methods known in the art can be used, e.g., ink jet printing, flexo printing, laser printing, thermo-transfer printing, pad printing, printing using cold lamination techniques, and the like.

In another embodiment, the indicator compound is part of a thermal transfer (TTR) ink composition and is transferred to the printed surface by applying heat to the TTR layer.

By means of a reference scale printed with the time-temperature integrator, absolute determination of quality grades is possible. The time-temperature integrator and the reference scale are advantageously arranged on a light-colored substrate in order to facilitate reading.

It is possible to apply, preferably in black ink, further text (or information), such as an expiry date, product identification, weight, contents etc.

The reference color may be changed as one means for changing the lifetime of the TTI.

The time-temperature indicator may be covered with a protective film, designed to avoid photo recharging and/or photo bleaching. The present invention relates especially to such a time temperature indicator further comprising a filter avoiding recharging or photobleaching of the time temperature indicator.

Either the TTI or the filter may be printed using cold lamination techniques or pad printing techniques.

The protective film is, for example, a color filter, e.g. a yellow filter, which are permeable only to light having typical wavelengths that are longer than 430 nm.

Suitable filters are disclosed in the International application WO2008049755. Disclosed therein is a composition comprising at least one ultraviolet light and/or visible light absorbing layer which is adhered to an underlying layer containing a photo-chromic colorant, which photo chromic colorant is activated by exposure to UV light to undergo a reversible color change, which color reversion occurs at a rate that is dependent on temperature, wherein the light absorbing layer comprises a binder,

from 1 to 60% by weight based on the total weight of the layer of an ultraviolet light absorber selected from the group consisting of hydroxyphenylbenzotriazole, benzophenone, benzoxazone, α-cyanoacrylate, oxanilide, tris-aryl-s-triazine, formamidine, cinnamate, malonate, benzilidene, salicylate and benzoate ultraviolet light absorbers.

If desired an irreversible photo-sensitive indicator can be applied as tamper-proofing in the form of a covering over the time-temperature integrator. Suitable irreversible indicators include, for example, pyrrole derivatives, such as 2-phenyl-di(2-pyrrole)methane. Such a material turns irreversibly red when it is exposed to UV light.

The invention further relates to a method of time temperature indication by converting the thioalkyl- or thioaryl-spiroaromatic indicator compounds of the formula I, Ia, II, III or IV as defined here in from an original stable state into a metastable state by a process selected from photonic induction, thermal induction, pressure induction, electrical induction, or chemical induction and detecting the time temperature dependent re-conversion from the metastable state to the original stable state.

The color change is preferably detected based on the color difference between said metastable and original state.

The time temperature detection may be achieved optically by detecting a change in an optical property (such as for example absorption, transmission, reflectivity) of the TTI device. For instance, a color change is determined either visually by comparing to a reference sample, or using a colorimeter or any colour reading or colour comparing technique.

The following Examples illustrate the invention.

### EXAMPLES

### Preparation of Alkylsulfanyl Derivatives of Spiropyrans

The photochromic spiropyran compounds of the present invention may be prepared according to synthetic routes known in the literature. Preparation of some oligomeric compounds was described by us earlier in WO2008090045A1.

### Example 1

### FPSP378

### 1-(4′-fluorobenzyl)-3,3-dimethyl-8″-methylsulfanyl-6″-nitro-spiro[2H-1-benzopyrane-2,2″-indoline]

### Preparation of 3-methylsulfanyl-5-nitro-salicylic aldehyde

Method A. Starting from Salicylic Aldehyde

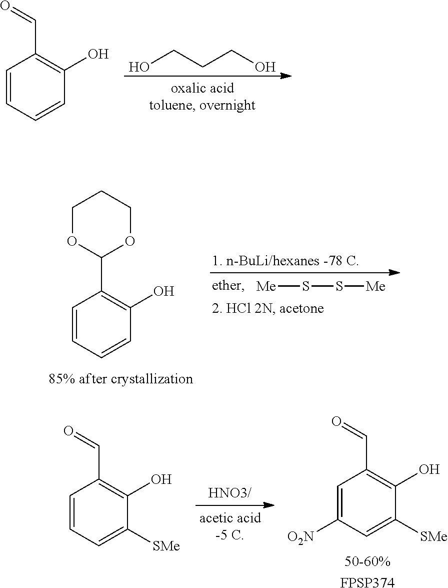

Method B Starting from 2-methylmercaptophenol

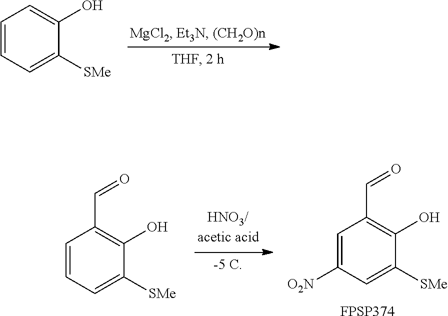

Method A. Step 1: 2-[1,3]dioxan-2-yl-phenol

A mixture of salicylic aldehyde (35 g, 287 mmol), oxalic acid (645 mg, 7.17 mmol, 0.025 eq) and 1,3-propanediol (32.7 g, 430 mmol, 31.1 ml) in toluene (100 ml) is refluxed for 48 h in an apparatus equipped with a condenser and a Dean-Stark trap. The reaction mixture is cooled to room temperature, washed with NaHCO₃, brine, dried over Na₂SO₄ and passed through alumina pad. The solvent is evaporated to dryness, a residue is dissolved in hot hexane, crystallized from hexane (under cooling), to afford 41.2 g (80%, two crops) of 2-[1,3]dioxan-2-yl-phenol as a white solid.

TLC: alumina-dichloromethane-hexane 1:1, R_{f}=0.3.

### Step 2: 3-methylsulfanyl salicylic aldehyde

To a mixture of 2-[1,3]dioxan-2-yl-phenol (cyclic acetal of salicylic aldehyde 372-2) (10 g, 55.5 mmol) and tetramethyl ethylene diamine (TMEDA, 32.2 g, 41.6 ml, 277 mmol) in dry ether (500 ml) at −78C under N₂ n-BuLi (198 ml, 277 mmol, a 1.4 M solution in hexanes) is added. The solution is allowed to warm up to room temperature (the reaction mixture is stirred overnight), during which time the initial precipitate dissolves and the solution slowly becomes orange. Dimethyl disulfide (31.4 g, 333 mmol, 30 ml) is then carefully added under cooling (mildly exothermic), giving a yellow solution and white precipitate which is stirred overnight. The reaction mixture is quenched with ammonium chloride (100 ml), the organic phase is separated, the aqueous phase is extracted with dichloromethane (2×100 ml), joined organic phases are washed with brine, dried over sodium sulfate, passed through alumina pad, evaporated to dryness. The protection group is removed using a mixture of acetone −2.5 M HCl (1:1) for ˜4 h. Then acetone is evaporated under reduced pressure, a product is extracted with dichloromethane, the organic phase is separated, dried over sodium sulfate, evaporated, to give 12 g of crude product. The crude is chromatographed on silica (hexane-ethyl acetate (0-5%)) to give rise to 6.4 g (68.6%) of TLC pure aldehyde 373 as yellow self crystallized oil.

TLC: silica: dichloromethane-hexane-1:1, R_{f}=0.3.

NMR, 300 MHz, (CDCl₃): δ, ppm-10.6 (s, 1H, broad, PhOH), 9.93 (s, 1H, CHO), 7.47 (d, 1H, J=7.6 Hz, Ar), 7.39 (d, 1H, J=7.8 Hz, Ar), 7.00 (t, 1H, J=7.5 Hz, Ar) 2.24 (s, 3H, S—CH₃).

### Step 3: 3-methylsulfanyl-5-nitro-salicylic aldehyde

A solution of 3-methylsulfanyl-salicylaldehyde (6.4 g, 38.0 mmol) in a mixture of acetic acid (50 ml) and CH₂Cl₂ (50 ml) is stirred in an ice bath at −10° C. Nitric acid 100% (11.99 g, 190 mmol, 7.9 ml, 5 eq) in 10 ml of acetic acid is added slowly by means of dropping funnel at such rate that the temperature is not exceed −5° C. The reaction mixture is stirred at −10° C. for 30 min. Then, the reaction mixture is poured into ice-water (300 ml) under vigorous stirring. The mixture is extracted with dichloromethane (3×30 ml), joined organic layers are washed with brine, dried over Na₂SO₄, passed through silica pad, and the solvent is evaporated to dryness. A residue is crystallized from ethanol to afford 4.0 g (49.3%) of the title compound as a yellow powder (TLC pure, two crops).

¹HNMR 300 MHz, (CDCl₃): δ, ppm-12.12 (s, 1H, PhOH), 9.93 (s, 1H, CHO), 8.29 (s, 1H, Ar), 8.14 (d, 1H, J=3 Hz, Ar), 2.51 (s, 3H, S—CH₃).

### Step 4: 1-(4′-fluorobenzyl)-3,3-dimethyl-8″-methylsulfanyl-6″-nitro-spiro[2H-1-benzopyrane-2,2″-indoline]

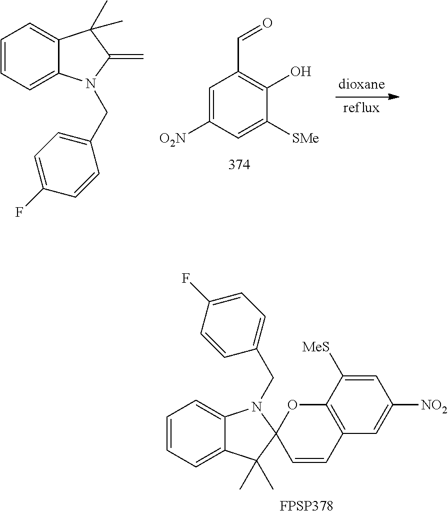

To a suspension of 3-methylsulfanyl-5-nitro-salicylic aldehyde (0.32 g, 1.5 mmol) in dioxane (5 ml) 1-(4′-fluorobenzyl)-3,3-dimethyl-2-methylene-indoline (0.38 g, 1.43 mmol) in dioxane (3 ml) is added dropwise under heating. The reaction mixture is refluxed for 1 h (TLC control), cooled to room temperature, evaporated to dryness, dissolved in dichloromethane, passed through alumina pad, crystallized from ethanol, to afford 0.33 g (50%) of FPSP378 as a yellow solid.

¹HNMR 300 MHz, (CDCl₃): δ, ppm 7.8 (s, 1H, Ar), 7.74 (s, 1H, Ar), 6.24 (d, 1H, J=7.8 Hz), 5.85 (d, 1H, J=10.5 Hz), 4.38 (d, 1H PhCH₂, J=16.2 Hz), 2.28 (s, 3H, SCH₃), 1.29 (s, CH₃), 1.24 (s, CH₃).

MS: MH⁺463.144.

### Example 2

### FPSP379

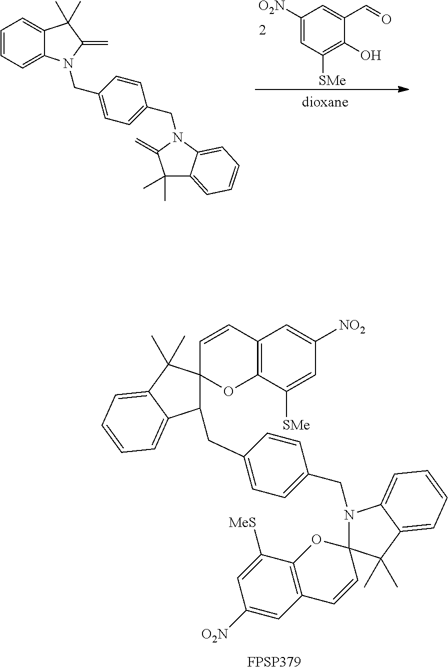

A solution of 1,4-bis((3,3-dimethyl-2-methyleneindolin-1-yl)methyl)benzene (prepared as described in WO2008090045A1, 1.0 g, 2.378 mmol) and 2-hydroxy-3-methylsulfanyl-5-nitrobenzaldehyde (prepared as described in Example 1, 1.1 g, 5.23 mmol, 2.2 eq) in dioxane (35 ml) is refluxed for 2 h. Then the reaction mixture is cooled to room temperature, evaporated to dryness, dissolved in mixture dichloromethane-hexane-2:1, passed through an alumina pad, evaporated to dryness.

A residue is triturated with ethanol, n-butanol (overnight), dried in vacuum to afford 0.8 g (43.7%) of FPSP379 as a grey powder.

¹H NMR (300 MHz), CDCl₃: δ, ppm 7.8 (s, 1H, Ar), 7.76 (s, 1H, Ar), 7.72 (s, 2H, Ar), 7.04 (4H, two triplets, Ar), 6.96 (2H, m Ar), 6.24 (d, 2H, double bond, J=7.8 Hz), 5.85 (d, 2H, double bond, J=9 Hz), 4.39 (d, 2H PhCH₂, J=16.2 Hz), 4.1 (d, 2H PhCH₂, J=16.2 Hz), 1.49 (s, 3H, SCH₃), 2.25 (s, 3H, SCH₃), 1.28 (s, 6H, 2 CH₃), 1.17 (s, 6H, 2 CH₃),

MS: MH⁺811.25

### Example 3

### 1-(4-methyl-[1,1′,4′,1″]terphenyl)-3,3-dimethyl-8′″-methylsulfanyl-6′″-nitro-spiro[2H-1-benzopyrane-2,2′″-indoline] (FPSP388)

### Step 1: 1-bromo-4-ethoxymethoxymethyl-benzene

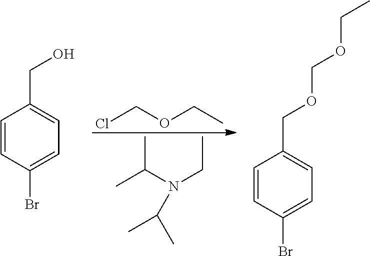

To a solution of 4-bromobenzyl alcohol (4 g, 21.39 mmol) and N,N-diisopropyl ethyl amine (4.7 g g, 36.4 mmol, 6.44 ml) in 50 ml of THF (dry, commercial) chloromethyl ethyl ether (2.426 g, 25.7 mmol, 2.4 ml) is added through syringe at 0° C. (under stirring and cooling with ice-water bath). The reaction mixture is stirred at 0° C. for 2 h, and then it is allowed to warm up to room temperature and stirred overnight. A white precipitate is filtered off, washed with THF. A filtrate is evaporated under reduced pressure to afford a crude product. The crude is dissolved in dichloromethane-hexane 1:1, passed through alumina pad, evaporated to dryness to give rise to the protected 4-bromobenzyl alcohol with quantitative yield (5.2 g).

### Step 2: 4-ethoxymethoxymethyl-[1,1′,4′,1″]terphenyl

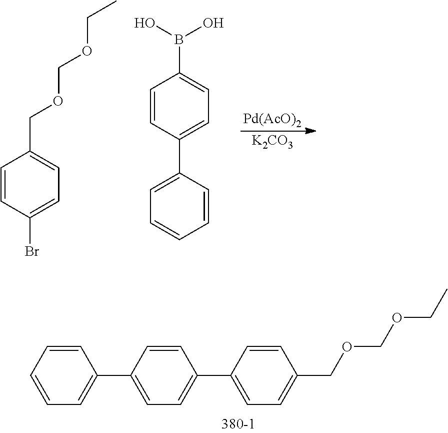

A mixture of 1-bromo-4-ethoxymethoxymethyl-benzene (3.4 g, 13.87 mmol, 1.eq), 4-biphenylboronic acid (3.85 g, 19.42 mmol, 1.4 eq), K₂CO₃ (5.75 g, 41.6 mmol, 3 eq), palladium acetate (16 mg, 0.069 mmol, 0.005 eq), pyridine (12 ml) are placed into dry round bottomed flask under nitrogen and heated to the reflux. After the reaction is completed (˜2 h) the reaction mixture is cooled to room temperature and the solvent was evaporated under reduced pressure. Pyridine traces are removed using azeotropic mixture with toluene. The residue is suspended in dichloromethane-hexane (1:1), passed through alumina pad. The solvents are evaporated; a white solid residue is triturated with minimal amount of hexane, filtered to afford 1.7 g (38.5%) of pure title compound.

TLC: alumina: dichloromethane-hexane 1:1. R_{f}=0.6

### Step 3: 4-bromomethyl-[1,1′,4′,1″]terphenyl

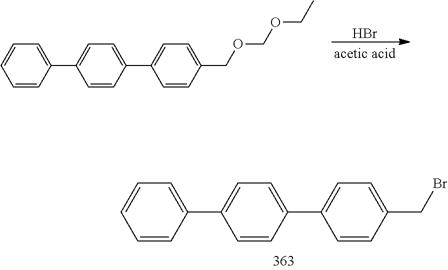

A suspension of 4-ethoxymethoxymethyl-[1,1′,4′,1″]terphenyl (2.6 g, 9.02 mmol) in HBr in acetic acid (33% HBr, 20 ml, 114 mmol, 12.67 eq) is heated at 130 C for 1.5 h, then the reaction mixture is cooled to room temperature, filtered, washed with hexane. A crude product is dried on a glass filter, recrystallized from ethyl acetate to afford 2.4 g, 91% (two crops) of 4-bromomethyl[1,1′,4′,1″]-terphenyl (FPSP363).

### Step 4: 1-(4-methyl-[1,1′,4′,1″]terphenyl)-3,3-dimethyl-2-methylene-indoline

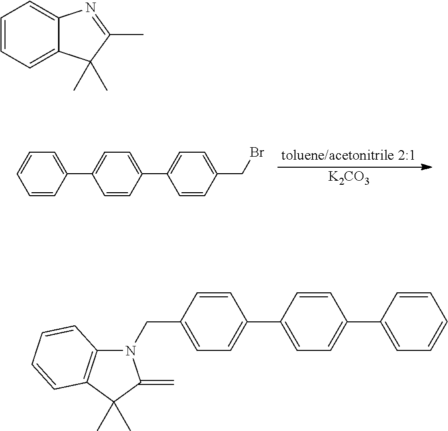

A mixture of 2,3,3-trimethyl-indolenine (1.675 g, 10.52 mmol, 1.7 ml), 4-(bromomethyl)-terphenyl (2 g, 8.09 mmol), potassium carbonate (2.9 g, 21.04 mmol) is heated at 90° C. in toluene-acetonitrile (12 ml, 2:1) for 48 h, cooled to room temperature. The reaction mixture is evaporated; a residue is partitioned between dichloromethane and 5% NaOH (aq). The organic layer is separated, water layer is back extracted with dichloromethane, joined organic phases are dried over Na₂SO₄, evaporated to dryness, to afford the crude product with quantitative yield. The product (contained the starting indolenine ˜25%) is subjected to the next step without delay.

### Step 5: 1-(4-methyl-[1,1′,4′,1″]terphenyl)-3,3-dimethyl-8′″-methylsulfanyl-6′″-nitro-spiro[2H-1-benzopyrane-2,2′″-indoline] (FPSP388)

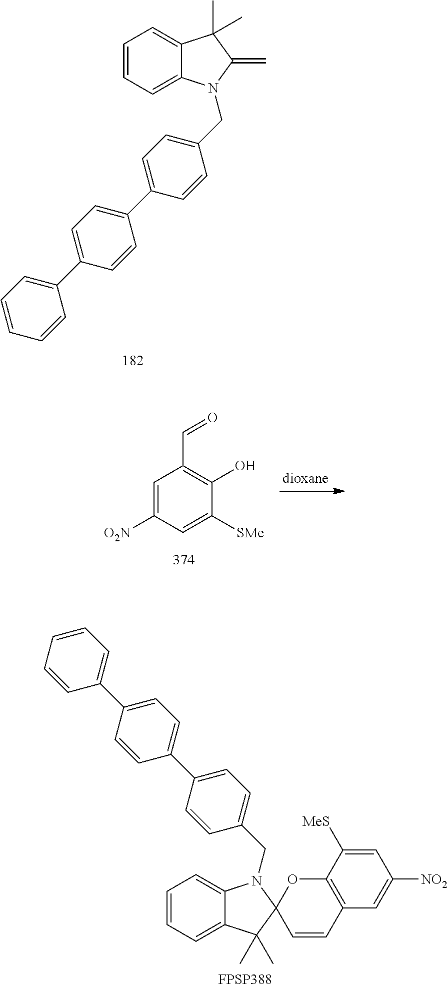

To a suspension of 3-methylsulfanyl-5-nitro-salicylic aldehyde (0.4 g, 1.88 mmol, prepared as described in Example 1) in 10 ml of dioxane 1-(4-methyl-[1,1′,4′,1″]terphenyl)-3,3-dimethyl-2-methylene-indoline (0.65 g, 1.62 mmol) is added under heating and stirring and the reaction mixture is refluxed 2 h. Then the reaction mixture is cooled to room temperature, evaporated to dryness, dissolved in mixture dichloromethane-hexane-1:1, passed through alumina pad, evaporated to dryness, crystallized from dichloromethane-ethanol, dried in vacuum, to afford 0.7 g (two crops, 63.8%) of FPSP388 as a light greenish powder.

TLC: alumina, dichloromethane-hexane-1:1, Rf=0.7.

### Example 4

### FPSP386 1-(4′-[1′,1″]-biphenyl)-3,3-dimethyl-8′″-methylsulfanyl-6′″-nitro-spiro[2H-1-benzopyrane-2,2′″-indoline]

### Step 1: 1-(4′-[1′,1″]-biphenyl)-3,3-dimethyl-2-methylene-indoline

The process of Step 4 in example 2 is followed with exception that commercial 4-bromomethylbiphenyl is used instead of 4-bromomethyl-[1,1′,4′,1″]terphenyl. The product is subjected to the next step without delay.

### Step 2: FPSP386 1-(4′-[1′,1″]-biphenyl)-3,3-dimethyl-8′″-methylsulfanyl-6′″-nitro-spiro[2H-1-benzopyrane-2,2′″-indoline]

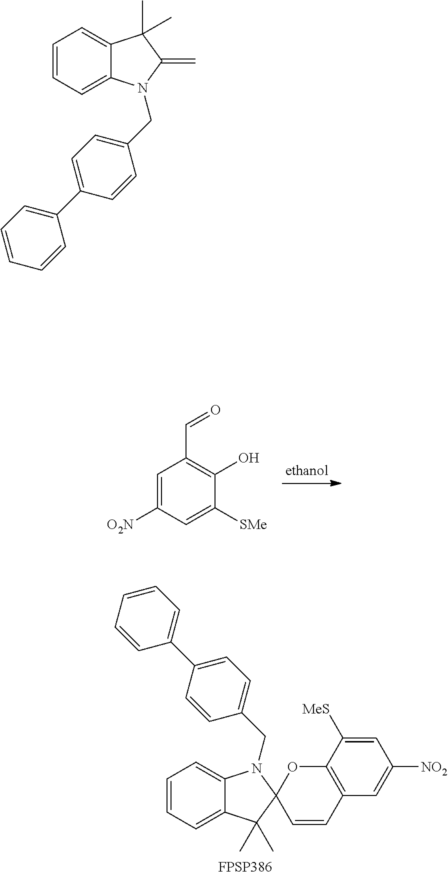

To a suspension of 3-methylsulfanyl-5-nitro-salicylic aldehyde (0.43 g, 2.03 mmol) in dioxane (15 ml) 1-(4′-[1′,1″]-biphenyl)-3,3-dimethyl-2-methylene-indoline (0.6 g, 1.84 mmol) in 5 ml of dioxane is added dropwise under stirring. The reaction mixture is refluxed for 1 h (TLC control), cooled to room temperature, evaporated to dryness, dissolved in dichloromethane, passed through alumina pad, crystallized from ethanol to afford 0.45 g (47%) of FPSP 386 as a yellow-greenish powder.

¹H NMR (500 MHz), CDCl₃: δ, ppm 7.86 (2H, d, J=7.5 Hz), 7.8 (s, 1H, Ar), 7.53 (2H, d, J=9.5 Hz), 7.31 (1H, d, J=7.5 Hz, Ar), 7.14 (1H, t, 6.9 (2H, m, Ar), 6.4 (1H, d, J=8 Hz, double bond), 5.97 (1H, d, J=10.5 Hz, double bond), 4.55 (1H, d, J=16.5 Hz, PhCH₂), 4.27 (1H, d, J=16.5 Hz, PhCH₂), 2.34 (s, 3H, SCH₃), 1.38 (3H, s, CH₃), 1.42 (3H, s, CH₃)

### Application-Examples

### Example 5

### Stabilization Against Photobleaching

Samples of the pigment were incorporated in identical water based ink, dispersed using a mill under the same conditions. The ink was printed on the same paper substance (LENETTA) and dried in an oven (30° C.) for 24 hrs. The samples were placed on 5 mm glass plates that served as a thermal reservoir and charged using the same light source (lamp 365 nm or LED 365—UV Light Emitting Diode (365 nm)). Two identical samples were prepared and charged from each ink. One system was placed in the dark at 0° C. while the other was exposed at 0° C. to filtered light (cutoff filter 455 nm) of a fluorescent lamp (“OSRAM” DULUX S G23, 900 lm, 11W/840), distance of 30 cm). The samples were measured using a colorimeter (Eye One GretagMacbeth). The CIE Lab values of the charged label that was kept in the dark were compared to the values of an identical label that was exposed to photobleaching light. As is evident from the following Table, methylthio groups consistently reduce the photosensitivity of the colored species.

Typically, the spiroaromatic compounds of the invention are incorporated into water based or solvent based ink (in some embodiments) prepared as follows.

### Preparation of the Ink Comprising Oligomeric Spiropyrans

Water based ink composition: 10% TTI

### Step 1: Polymer Matrix Preparation:

20 g of LS-16 (Ciba®GLASCOL® LS16—an aqueous microemulsion based on a carboxylated acrylic copolymer)
Joncryl74 (BASF resins—an aqueous microemulsion based on a carboxylated acrylic copolymer)
0.25 g of TEGO—TEGO® FOAMEX 845 defoamer emulsion of an organically modified polysiloxane, contains fumed silica)
0.1 g of triethanolamine (TEA)—stir for 1 minute

### Step 2: Preparation of the Ink Sample

0.2 g of TTI
1.6 g of the Polymer matrix
0.4 g of water (HPLC grade)

The mixture was dispersed on pulverisette (six cycles of 5 min at 600 rpm, twice: six cycles of 5 min at 800 rpm) to give the 10% TTI ink.

### Photobleaching Table at 0° C.

The CIE Lab values of the charged label that was kept in the dark at 0° C. were compared to the values of an identical label that was exposed at 0° C. to photobleaching light.

| | (L² + a² + b²)^{0.5} | Charging | Time, | (L² + a² + b²)^{0.5} | |
|---|---|---|---|---|---|
| Compound | uncharged | conditions | hrs | filter | Dark |
| 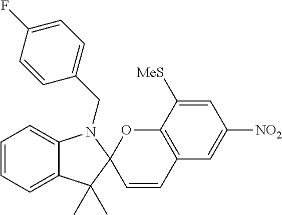 FPSP378 | 95 | 15 sec **LED 365 | 0 20 40 70 100 140 160 | 64 67 74 77 79 82 84 | 64 65 67 70 73 74 74 |
| 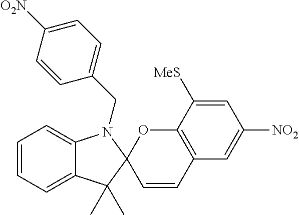 FPSP387 | 96 | 15 sec **LED 365 | 0 20 40 90 110 | 80 88 91 92 93 | 80 86 88 90 90 |
| 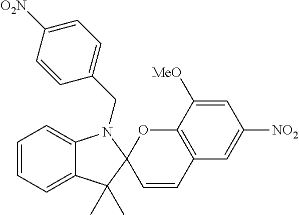 FPSP122 Comparative WO2008083925 | 94 | 15 sec **LED 365 | 0 20 50 70 100 120 170 | 58 67 70 73 76 79 82 | 58 65 68 68 69 70 73 |
| 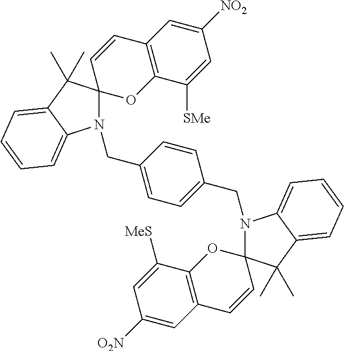 FPSP379-05-08 | 87 | 15 sec **LED 365 | 0 20 50 70 100 140 160 | 57 55 56 57 57 58 58 | 57 55 55 57 57 58 58 |
| 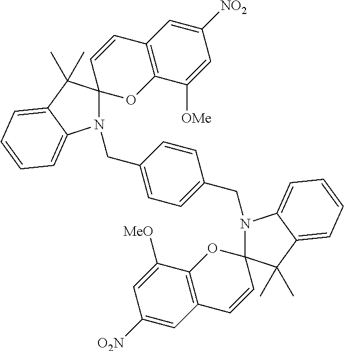 FPSP127 Comparative (WO08090045) | 81 | *3 min Tube lamp 365 nm | 0 50 70 100 120 160 | 51 55 57 59 61 63 | 51 54 55 55 55 56 |
| 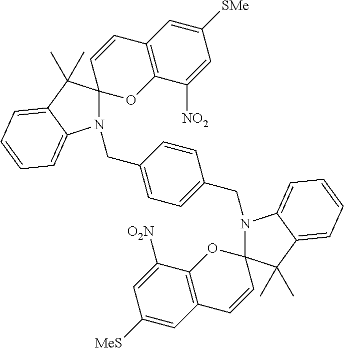 FPSP392 | 87 | 15 sec **LED 365 | 0 30 50 100 120 140 | 65 67 69 74 76 77 | 65 65 65 66 67 67 |
| 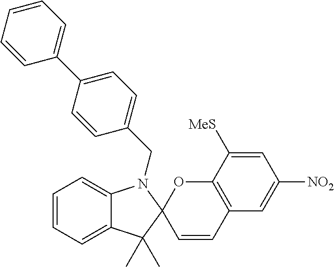 FPSP386 | 92 | 15 sec **LED 365 | 0 20 40 60 90 120 160 | 62 65 71 76 79 81 82 | 62 65 69 71 74 75 75 |
| 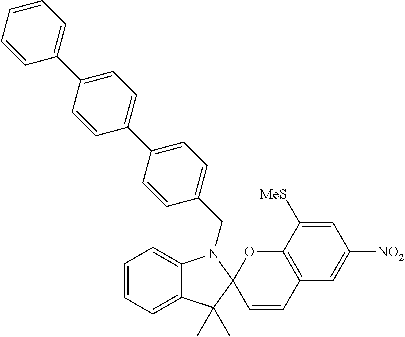 FPSP388 | 87 | 15sec **LED 365 | 0 20 40 60 100 120 160 | 55 52 53 53 54 54 55 | 55 52 53 53 54 54 55 |
| 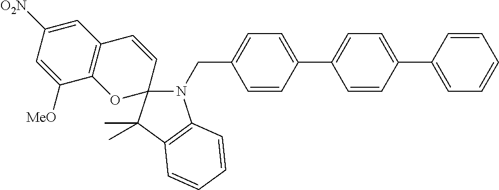 361 Comparative WO08083925 | 84 | 15sec **LED 365 | 0 20 40 70 100 120 160 | 52 51 50 50 50 50 51 | 52 54 54 54 54 55 55 |

Laboratory UV tube lamp VL-6.LC (6W −365 nm)
**LED 365—UV Light Emitting Diode (365 nm)

The preferred indicator compounds FPSP379, i.e. FPSP379-05-08, and FPSP388 practically have no photobleaching (see photobleaching data presented in Table 1).

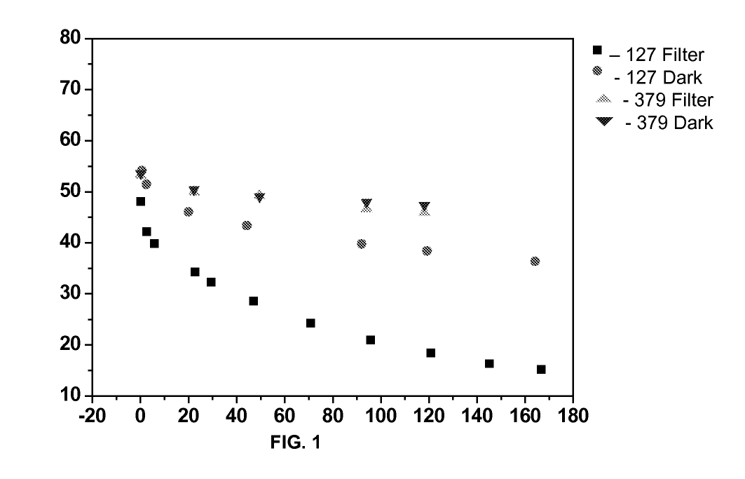

The fact that the difference between the sample kept in the dark and the filter sample is much reduced for the thioalkyl- or thioaryl-spiropyrans of the present invention in comparison to the corresponding spiropyrans wherein the sulphur atom(s) are replaced by oxygen atom(s) is also evident from FIGS. 1 to 4.

FIGS. 1 to 4 show along the horizontal axis the time [hours] and along the vertical axis the square root of the sum of the square delta Lab values, which is calculated from the CIE L, a, and b values according to the formula (ΔL²+Δa²+Δb²)^{0.5}, wherein ΔL is L₀−L, Δa is a₀−a and Δb is b₀−b and L₀, a₀ and b₀ are the values at time 0, i.e. the time just before charging.

FIG. 1 compares compound FPSP379-05-08 with FPSP127 (disclosed in WO08090045).

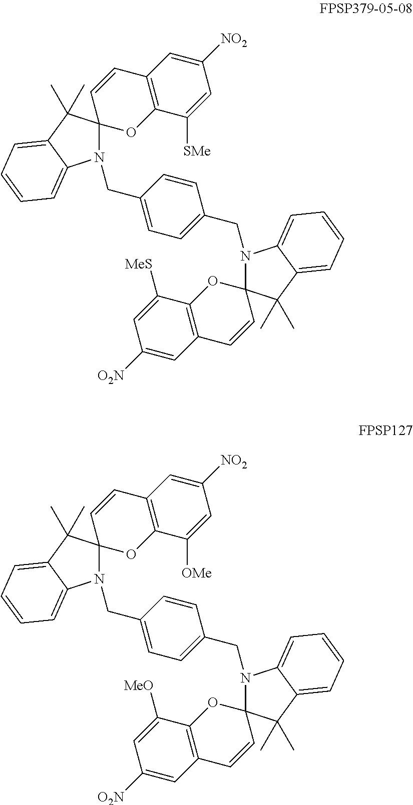

As evident from FIG. 1, the curves (filter or dark) for FPSP379-05-08 are nearly identical, whereas the curves (filter or dark) for FPSP127 differ largely from each other reflecting increased photobleaching of the filter sample in comparison to the sample kept in the dark.

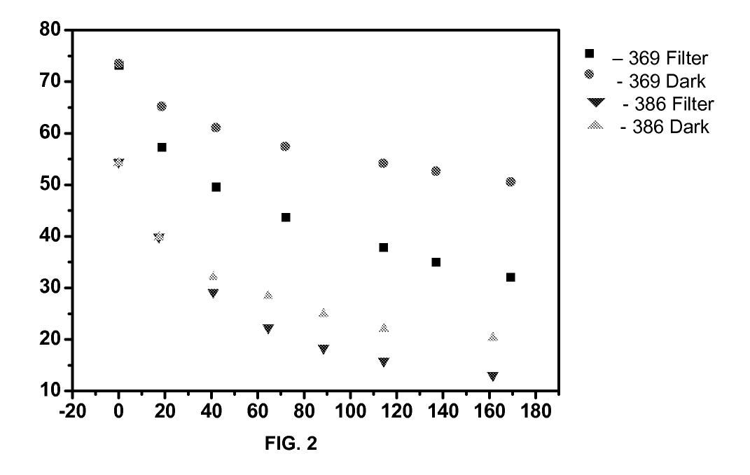

FIG. 2 compares compound FPSP386 with FPSP369.

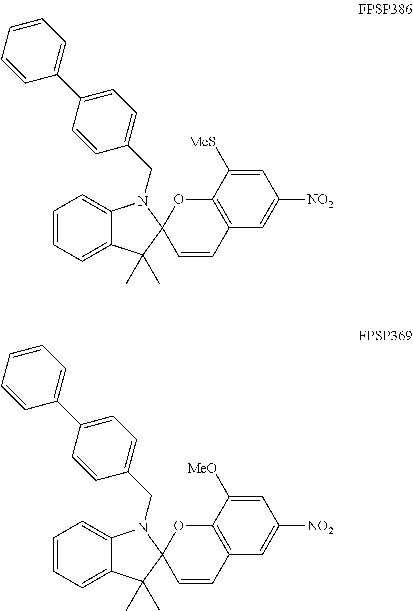

As evident from FIG. 2 the curves (filter or dark) for FPSP386 differ much less from each other than the curves (filter or dark) for FPSP369 reflecting increased photobleaching of the filter sample of FPSP369 in comparison to the sample kept in the dark.

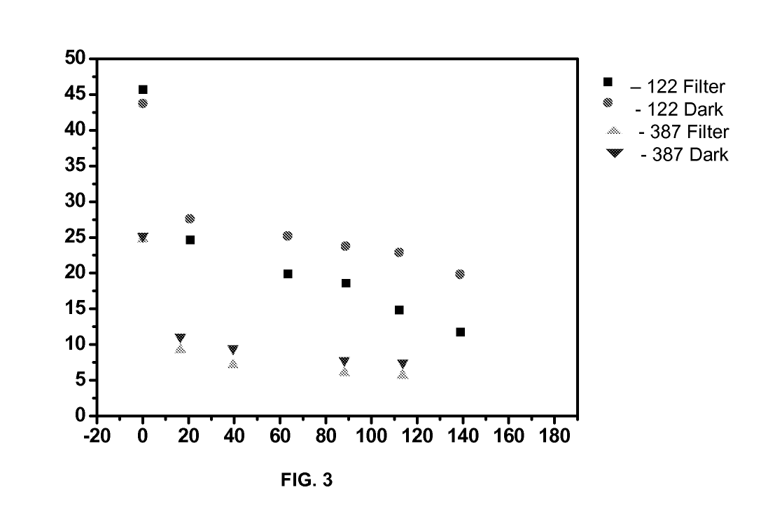

FIG. 3 compares compound FPSP387 with FPSP122 (disclosed in WO2008083925).

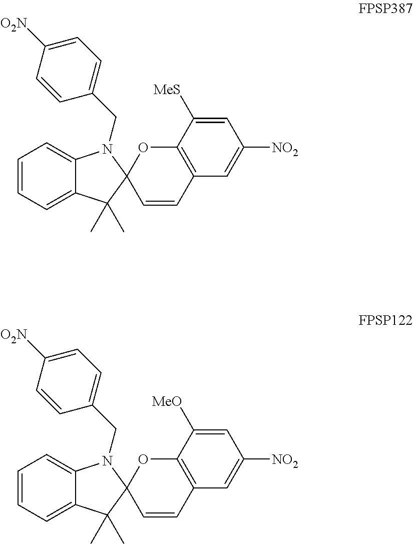

As evident from FIG. 3 the curves (filter or dark) for FPSP387 are nearly identical, whereas the curves (filter or dark) for FPSP122 differ largely from each other reflecting increased photobleaching of the filter sample in comparison to the sample kept in the dark.

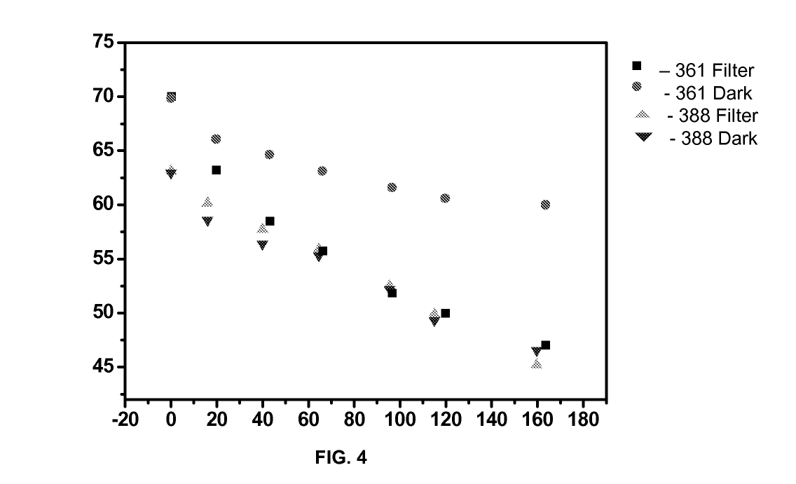

FIG. 4 compares compound FPSP388 with compound 361 (disclosed in WO08083925).

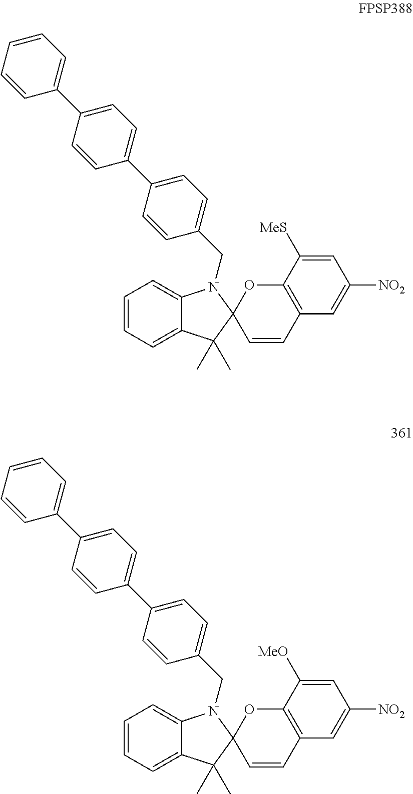

As evident from FIG. 4 the curves (filter or dark) for FPSP388 are nearly identical, whereas the curves (filter or dark) for compound 361 differ largely from each other reflecting increased photobleaching of the filter sample in comparison to the sample kept in the dark.

1. A time temperature indicator comprising at least one thioalkyl- or thioaryl-derivative of spiropyrans of the general formula I 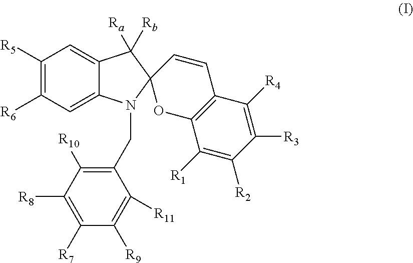
wherein
R₁-R₄ independently of one another is hydrogen, C₁-C₆ alkylsulfanyl, arylsulfanyl, halogen, CF₃, —C₁-C₆ alkyl or —NO₂ with the proviso that at least one of R₁-R₄ is C₁-C₆ alkylsulfanyl or arylsulfanyl;
R₅ is hydrogen, halogen, —C₁-C₆ alkoxy, —COOH, —COO—C₁-C₆alkyl, —CF₃ or phenyl;
R₆ is hydrogen or R₆ and R₅ form together a phenyl ring;
Rₐ is hydrogen or, —C₁-C₆ alkyl;
R_{b} is hydrogen or, —C₁-C₆ alkyl, or together with Rₐ form a 5-6 membered cycle;
R₇ is —C₁-C₆ alkoxy, —NO₂, —CF₃, ₋O—CF₃, —CN, —COO—C₁-C₆alkyl, phenyl or biphenyl;
R₈ is hydrogen, halogen, —CN, —C₁-C₆ alkoxy or R₇ and R₈ form together a phenyl ring;
R₉ is hydrogen, halogen, —CN, or —C₁-C₆ alkoxy;
R₁₀ is hydrogen or halogen or CN;
R₁₁ is hydrogen or halogen or CN.

2. A time temperature indicator according to claim 1, wherein
R₁ is C₁-C₆ alkylsulfanyl, arylsulfanyl;
R₂ is R₄ is hydrogen;
R₃ is NO₂,
R₅ and R₆ are hydrogen;
Rₐ and R_{b} are methyl;
R₇ is —C₁-C₆ alkoxy, —NO₂, —CF₃, ₋O—CF₃, —CN, —COO—C₁-Colkyl, phenyl or biphenyl
R₈ and R₉ and R₁₀ and R₁₁ are hydrogen.

3. A time temperature indicator comprising at least one thioalkyl- or thioaryl-derivative of the formula Ia 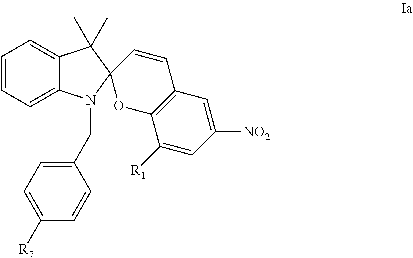
wherein R₁ is MeS or arylsulfanyl and R₇ is hydrogen or halogen.

4. A time temperature indicator comprising at least one thioalkyl- or thioaryl-derivatives of spiropyrans of the general formula II or III 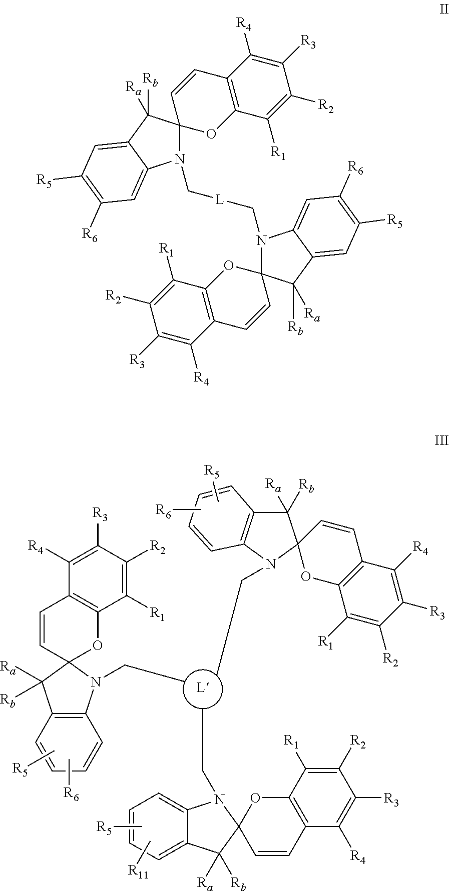
wherein
R₁-R₄ independently of one another is hydrogen, C₁-C₆ alkylsulfanyl, arylsulfanyl, halogen, CF₃, —C₁-C₆ alkyl or —NO₂ with the proviso that at least one of R₁-R₄ is C₁-C₆ alkylsulfanyl or arylsulfanyl;
R₅ is hydrogen, halogen, —C₁-C₆ alkoxy, —COOH, —COO—C₁-C₆ alkyl, —CF₃ or phenyl;
R₆ is hydrogen or R₆ and R₅ form together a phenyl ring;
Rₐ is hydrogen or, —C₁-C₆ alkyl;
R_{b} is hydrogen or, —C₁-C₆ alkyl, or together with Rₐ forms a 5-6 membered ring
L is a divalent linker;
L′ is a trivalent linker.

5. A time temperature indicator according to claim 4, wherein the thioalkyl- or thioaryl-derivatives of spiropyrans is a compound of the formula II wherein
R₁ is —C₁-C₆ alkylsulfanyl, arylsulfanyl;
R₂ is R₄ is hydrogen;
R₃ is NO₂
R₅ and R₆ are hydrogen;
Rₐ and R_{b} are methyl;
L is a divalent linker.

6. The time-temperature indicator according to claim 1, wherein the at least one thioalkyl derivative of the spiropyran indicator compound is selected from the group consisting of the following structural formulae

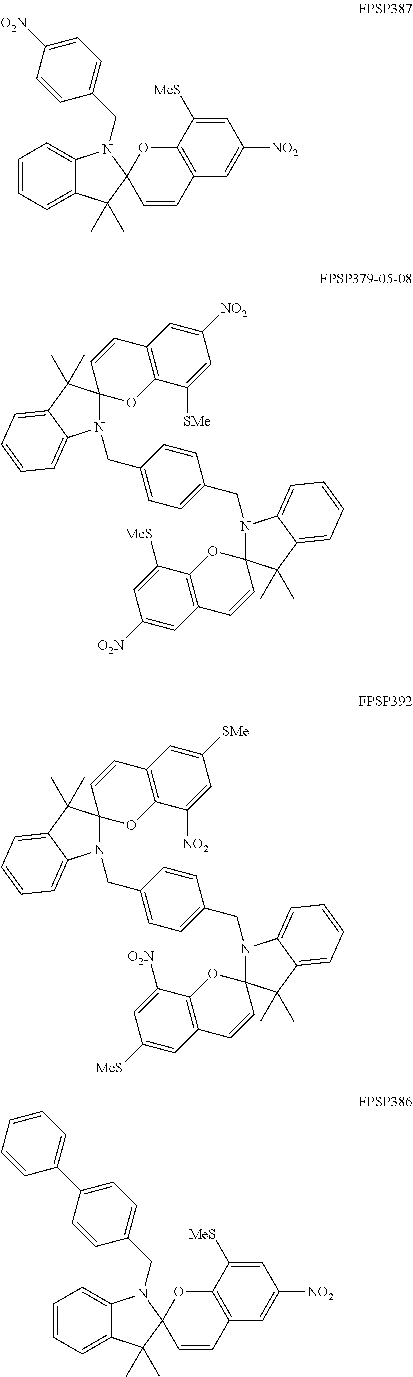

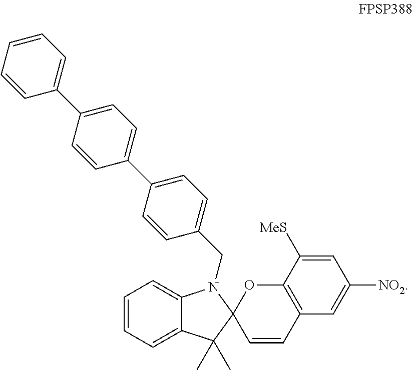

7. A time temperature indicator according to claim 1 further comprising a filter avoiding recharging or photobleaching of the time temperature indicator.

8. A method of manufacturing a time-temperature indicator comprising at least one thioalkyl- or thioaryl-derivatives of spiropyrans of the general formula I, Ia, II, III or IV in form of a pigment or a dye; said method comprising the steps of
(a) introducing into a matrix or atop a matrix a thioalkyl- or thioaryl-derivatives of spiropyrans of the general formula I, Ia, II, III or IV as defined in claim 1, and
(b) converting the spiropyran indicator from an original stable state into a metastable state by a process selected from photonic induction, thermal induction, pressure induction, electrical induction, or chemical induction,
(c) optionally applying a protector film.

9. A method of time temperature indication by converting the thioalkyl- or thioaryl-derivatives of spiropyrans of the general formula I, Ia, II, III or IV as defined in claim 1 from an original stable state into a metastable state by a process selected from photonic induction, thermal induction, pressure induction, electrical induction, or chemical induction and detecting the time temperature dependent re-conversion from the metastable state to the original stable state.

10. The method of claim 9, wherein a color change is detected based on the color difference between said metastable and original state.

11. A printing ink or printing ink concentrate, comprising at least one thioalkyl- or thioaryl-derivatives of spiropyrans of the general formula I, Ia, II, III or IV as defined in claim 1; for manufacturing a time temperature indicator.
